(19) United States
(12) Patent Application Publication
(10) Pub. No.: US 2014/0200647 A1
(43) Pub. Date: Jul. 17, 2014

(54) STENT WITH ENHANCED PROFILE

(71) Applicant: ABBOTT CARDIOVASCULAR SYSTEMS INC., Santa Clara, CA (US)

(72) Inventor: Chad J. Abunassar, San Francisco, CA (US)

(73) Assignee: ABBOTT CARDIOVASCULAR SYSTEMS INC., Santa Clara, CA (US)

(21) Appl. No.: 13/741,300

(22) Filed: Jan. 14, 2013

(51) Int. Cl. A61F 2/95; A61F 2/958

(52) U.S. Cl. CPC A61F 2/95; A61F 2/958
USPC 623/1.11

(57) ABSTRACT

A stent for implantation in a body lumen, comprising a plurality of rings, each ring being connected to an adjacent ring by at least one link, each ring including a plurality of peaks and valleys, wherein each peak is connected to an adjacent valley by a strut to provide an undulating pattern within each ring. Further, wherein each of a plurality of the struts comprise a first portion and a second portion; each portion has a thickness that is substantially constant throughout both first and second portions; and each portion has a width that is substantially constant throughout both first and second portions. The first portion is connected to the second portion through a reduced zone positioned at a mid-point of the strut, wherein the reduced zone has a minimum thickness between 30% and 80% of the thickness of the first and second portions.

### BACKGROUND

The present invention relates to stent and scaffold design for placement in a body lumen of a patient. More specifically, the invention relates to a novel method for dynamically enhancing the profile of a stent to reduce injury caused by structural corners that develop in an expanding stent.

Stent and scaffold design for placement in a body lumen is a field that is well known in the art. Metallic or polymeric stents or scaffolds are known for their ability to hold open body lumens that are prone to collapse, or that have suffered an injury and require support. Stents are typically formed from a resilient metal or polymer, and are typically cut from a tube using laser energy with a highly focused degree of precision. As may be understood with reference to FIG. 1 and FIG. 2, many stents are configured to include a series of rings 10 which fit within a generally annular profile, but which follow an undulating path formed by peaks 12 and valleys 14 that are joined to each other by elongate struts 16 to form an undulating pattern that extends around the circumference of the stent. Each ring is connected to an adjacent ring by at least one link 18, and such links may have a number of shapes and configurations in various stents.

The typical stent known in the art thus described has a first condition in which it has a compressed first diameter (exemplified in FIG. 1), suitable for installing on the tip of a catheter for insertion into the body lumen by known minimally invasive techniques. Once the stent is delivered to a desired location in the body lumen in the first condition, the stent is expanded to a second condition, as exemplified in FIG. 2 and FIG. 4A. This may be accomplished either by forming the stent to possess self-expansive metallurgical or polymeric properties which come into play once a confining sheath is withdrawn from the stent, or by inflating a balloon positioned within the bore of the stent, or by a combination of both. As a result, the rings 10 of the stent adjust their shape to assume an expanded diameter suitable for supporting an inner wall of the body lumen against collapse, as exemplified in FIG. 2 and FIG. 4A.

However, although expandable stents have now played a major role in vessel treatment over a number of years, problems associated with stent implantation have been identified. Typically, stents in the second expanded condition as described have corners or bends at the location of the peaks and valleys. As is apparent in FIG. 4A, the corner extremities of the stent 10 act to cause a sharp change in direction of the tissue T surrounding the stent as the tissue passes over a terminal end (peak or valley) of the stent. Stated differently, the tissue T experiences a change of direction with the smallest radius of curvature at the terminal corners of the ring 10. Those of ordinary skill in the art will understand that the pressure profile experienced by the vessel along the length of the ring is highest at the location of the terminal corners, as indicated in FIG. 4B which schematically plots the pressure profile in the tissue along the length of a ring. The result is that these corners tend to dig into the body lumen tissue T. This effect may cause localized vessel injury within the span of the stented region, and may induce a propensity for inflammation, restenosis, and thrombosis.

Thus there exists a need in the art for a stent configuration that will overcome the disadvantages of the prior art. The present invention addresses these and other needs.

### SUMMARY OF THE INVENTION

In some embodiments, the invention comprises a system for treating a vascular condition, typically a condition in a human being. The system comprises a catheter configured for insertion into a vessel such as a vein or an artery. A stent mounted is on the catheter and configured for delivery into the vessel. The stent comprises a plurality of rings, each ring being connected to an adjacent ring by at least one link, each ring including a plurality of peaks and valleys, wherein each peak is connected to an adjacent valley by a strut to provide an undulating pattern within each ring. Each of a plurality of the struts comprises, in series, a first portion which is directly connected to a second portion, the second portion being directly connected to a third portion. Each of the first portion and the third portion has a thickness that is substantially constant throughout both the first portion and the third portion. The second portion has a minimum thickness between 30% and 80% of the thickness of the first and third portions, and further wherein the second portion is not coated with any therapeutic material.

In some embodiments, the stent may be a balloon expandable stent, while in other embodiments, the stent may be a self-expanding stent. In further embodiments, the second portion has a minimum thickness between 45% and 65% of the thickness of the first and third portions. Furthermore, in some embodiments, the second portion may extend for a length not more than three times the thickness of the first and third portions.

In some embodiments, each of the first portion and the third portion has a width that is substantially constant throughout both the first portion and the third portion, and the second portion may have a minimum width that is between 30% and 80% of the width of the first and third portions. In other embodiments, the second portion may have a minimum width that is between 45% and 65% of the width of the first and third portions. In some embodiments, the second portion is positioned on a strut midway between a valley and a peak.

In another embodiment, the invention is a system for treating a vascular condition. The system comprises a catheter configured for insertion into a vessel. A stent is mounted on the catheter and configured for delivery into the vessel. The stent comprises a plurality of rings, each ring being connected to an adjacent ring by at least one link, each ring including a plurality of peaks and valleys, wherein each peak is connected to an adjacent valley by a strut to provide an undulating pattern within each ring; and further wherein each of a plurality of the struts comprises, in series, a first portion which is directly connected to a second portion, the second portion being directly connected to a third portion; each of the first portion and the third portion has a thickness that is substantially constant throughout both the first portion and the third portion; and each of the first portion and the third portion has a width that is substantially constant throughout both the first portion and the third portion; and the second portion extends for a length not more than three times the thickness of the first and third portions, and wherein the second portion has a minimum width that is between 30% and 80% of the width of the first and third portions, and further wherein the second portion is not coated with any therapeutic material.

In some embodiments, stent is a balloon expandable stent, in other embodiments the stent is a self-expanding stent. In some embodiments, the second portion has a minimum width that is between 45% and 65% of the width of the first and third portions. In some embodiments, the second portion has a minimum thickness that is between 30% and 80% of the thickness of the first and third portions, and in other embodiments the second portion has a minimum thickness that is between 45% and 65% of the thickness of the first and third portions. Preferably, the second portion is positioned on a strut midway between a valley and a peak.

In another embodiment, the invention is a method of treating a condition in a vessel. The method comprises mounting a stent on a catheter, wherein the stent includes a plurality of rings, each ring being connected to an adjacent ring by at least one link, each ring including a plurality of peaks and valleys, wherein each peak is connected to an adjacent valley by a strut to provide an undulating pattern within each ring, and further wherein each of a plurality of the struts comprises, in series, a first portion which is directly connected to a second portion, the second portion being directly connected to a third portion; and further wherein each of the first portion and the third portion has a thickness that is substantially constant throughout both the first portion and the third portion; wherein the second portion has a minimum thickness between 30% and 80% of the thickness of the first and third portions, and further wherein the second portion is not coated with any therapeutic material. The stent is delivered to a desired location in the vessel, whereafter it is deployed to an expanded condition inside the vessel. Each of the plurality of struts is caused to bend radially outwardly at the location of the second portion by causing plastic deformation outwards in the second portion while causing no plastic deformation outwards in the first and third portions.

In some embodiments, causing plastic deformation outwards in the second portion includes causing plastic deformation outwards over a strut length that does not exceed three times the thickness of the first and third portions. In some embodiments, deploying the stent to an expanded condition includes deploying the stent by balloon, and in other embodiments, it includes allowing the stent to self expand. Preferably, causing each of the plurality of struts to bend radially outwardly includes causing each of the plurality of struts to bend radially outwardly at a location midway between a valley and a peak.

In another embodiment, the invention is a method of treating a condition in a vessel. The method comprises mounting a stent on a catheter, wherein the stent includes a plurality of rings, each ring being connected to an adjacent ring by at least one link, each ring including a plurality of peaks and valleys, wherein each peak is connected to an adjacent valley by a strut to provide an undulating pattern within each ring; each of a plurality of the struts comprises, in series, a first portion which is directly connected to a second portion, the second portion being directly connected to a third portion; each of the first portion and the third portion has a thickness that is substantially constant throughout both the first portion and the third portion; each of the first portion and the third portion has a width that is substantially constant throughout both the first portion and the third portion; the second portion extends for a length not more than three times the thickness of the first and third portions, and wherein the second portion has a minimum width that is between 30% and 80% of the width of the first and third portions, and further wherein the second portion is not coated with any therapeutic material. Thereafter, the stent is delivered to a desired location in the vessel, and is deploying to an expanded condition inside the vessel. Each of the plurality of struts is caused to bend radially outwardly at the location of the second portion by causing plastic deformation outwards in the second portion while causing no plastic deformation outwards in the first and third portions. In some embodiments deploying the stent to an expanded condition includes deploying the stent by balloon, and in other embodiments it includes allowing the stent to self expand. In a preferred embodiment, causing each of the plurality of struts to bend radially outwardly includes causing each of the plurality of struts to bend radially outwardly at a location midway between a valley and a peak.

When read in light of the drawings and the detailed description of the preferred embodiments, these and other advantages will be apparent.

### BRIEF DESCRIPTION OF THE DRAWINGS

FIG. 1 is a schematic “roll-out” view (i.e., a flat view of a cylindrical surface) of a stent of a variety known in the prior art, shown in an unexpanded condition.

FIG. 2 is a perspective view of a ring component of the stent in FIG. 1, shown in an expanded condition.

FIG. 3 is a perspective view of a component of a stent having features of the present invention, shown in an expanded condition.

FIG. 4A is a sectional view, taken substantially along the line A-A in FIG. 2, of a stent of known variety in the prior art that is placed within a body lumen.

FIG. 4B is a schematic plot, taken along the stent in FIG. 4A, of maximum pressure experienced by a vessel when surrounding the stent in FIG. 4A.

FIG. 5A is a sectional view, taken substantially along the line B-B in FIG. 3, of a stent of having features of the present invention that is placed within a body lumen.

FIG. 5B is a schematic plot, taken along the stent in FIG. 5A, of maximum pressure experienced by a vessel when surrounding the stent in FIG. 5A.

FIG. 6 is a plan view of a detail of a stent having features of the present invention.

FIG. 7 is a side elevation view of a detail of a stent having features of the present invention.

FIG. 8 is a plan view of a detail of a stent having features of the present invention.

FIG. 9 is a sectional view taken substantially along the line C-C in FIG. 7.

FIG. 10 is a plot result of radial strength, obtained by bench test assessment, of (a) an unmodified stent, and (b) a modified stent that includes features of the present invention.

FIG. 11 is a plot result of recoil, obtained by bench test assessment, of (a) an unmodified stent, and (b) a modified stent that includes features of the present invention.

FIG. 12 is a plot result of vessel injury, obtained by finite element analysis caused by (a) an unmodified stent, and (b) a modified stent that includes features of the present invention.

### DETAILED DESCRIPTION OF THE PREFERRED EMBODIMENTS

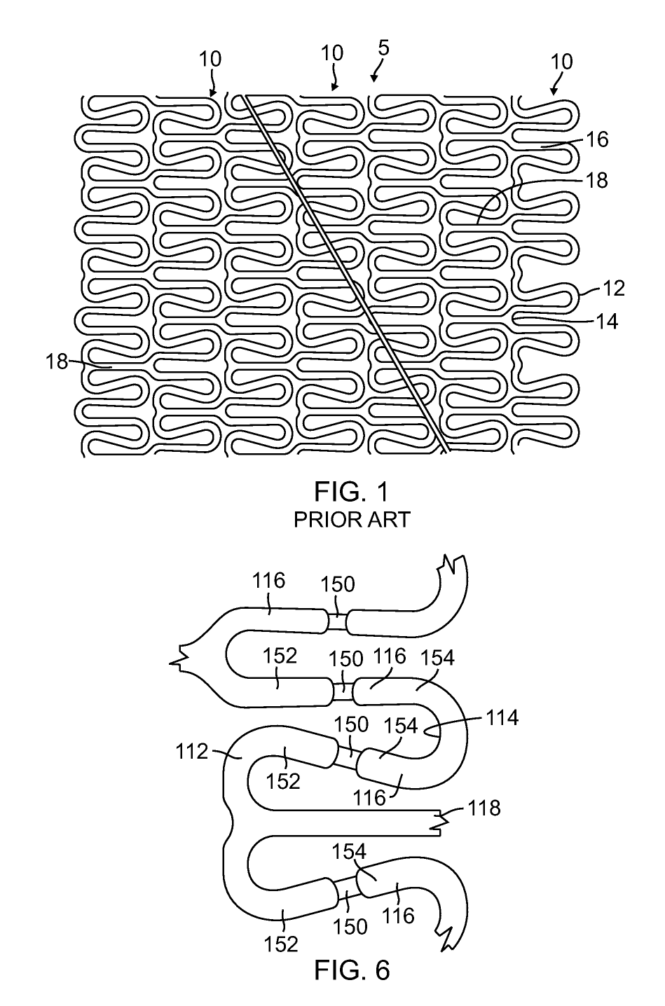

With reference to FIGS. 3, 5A-5B, and 6-9, embodiments of the invention are described. The invention is a system for vascular treatment that comprises a stent which may be formed from metal or polymer. The stent may be manufactured by cutting it from a tube using known laser cutting technology. The stent is configured to include a series of rings 110 which fit within a generally annular profile, but which follow an undulating path formed by peaks 112 and valleys 114 that are joined to each other by elongate struts 116 to form an undulating pattern that extends around a circumference. When the rings 110 are combined in series according to a pattern of the kind seen in FIG. 1, each ring is connected to an adjacent ring by at least one link. The stent 110 may be balloon expandable, or self-expanding.

Structure Unique to Invention

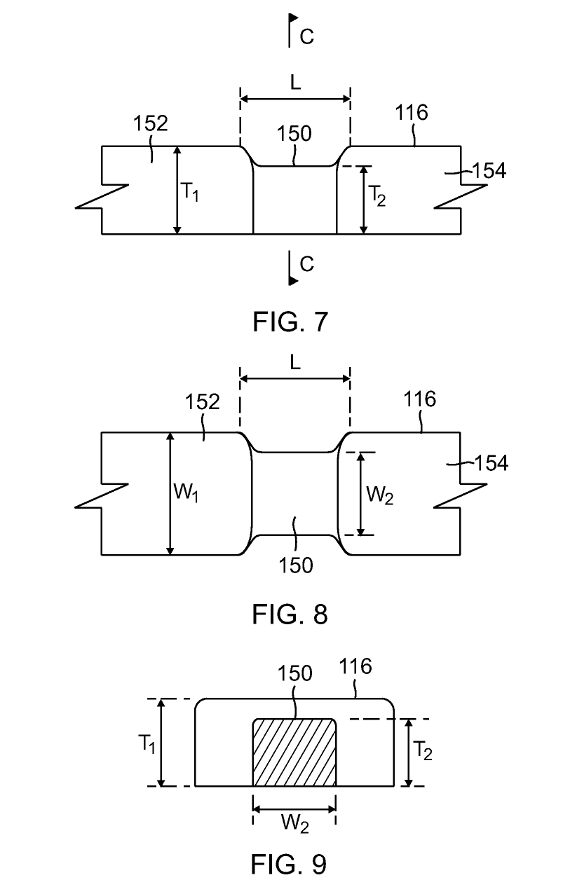

In a preferred embodiment of the invention, each strut 116 may be shaped, at its mid-point between peak and valley to include what will be termed herein as a “pin joint.” The pin joint 150 includes a structurally reduced zone, in which the radial thickness dimension of the strut is reduced to a thinner dimension, as shown in detail in FIGS. 7-9. Thus, the strut 116 may be divided into three serially connected portions, a first portion 152, a second portion or pin-joint 150, and a third portion 154—as indicated in FIGS. 6-9. For example, while the stent may be cut from a tube that has an overall thickness T1, thereby imparting overall strut thickness T1 to the struts of the stent, the localized thinning at the pin joint may include a thickness T2 that is less than T1, and preferably between about 30% to 80% of T1, even more preferably between 45% and 65% of T1. The thinning is preferably localized within a length L that is between one and three times the general thickness T1 of the strut. By holding the length L to a relatively short length as described, the effect of the thinned portion is to provide a pin type joint that will cause bending deformation in the strut to manifest primarily at the pin joint, and thereby to localize bending in the strut to occur at the location of the pin joint. The advantage of this effect will be described more fully below. It may be noted however, that a feature of the pin joint as described is that the space on the strut that is formed by the pin joint is not filled in with any material that is of a different substance than that from which the structural portion of the strut is formed. For example, the pin joint is left free of any material coating that may be designed for chemically therapeutic purposes, such as a coating which is impregnated with a drug, therapeutic substance, or active agent. If the reduced area forming the pin joint were to be filled in with such a material, the advantageous effects of the invention as described herein would not be immediately available upon deployment of the stent in a vessel and an advantage of the invention would be lost.

In some further embodiments, the strut may be shaped to include a local narrowing in the width of the strut, measured circumferentially across the surface of the strut, as exemplified in FIGS. 6-9. Thus, although the strut may be cut to have a substantially constant width W1 over its length, in the region of the pin joint, and more particularly over the length L as described above, the width may be symmetrically narrowed to W2. (As used herein, the term “substantially” in relation to a dimension or location means within manufacturing tolerance, plus 75% of manufacturing tolerance.) Thus, in some embodiments at the location of the pin joint the strut may have a reduced dimension in both the radial and circumferential directions. In a preferred embodiment, the narrowed width W2 may be between 30% and 80% of the original width W1 of the strut. In further more preferred embodiments, the narrowed width W2 may be between 45% and 65% of the original width W1 of the strut. Narrowing the strut will tend to contribute to the advantageous behavior of the stent that is described below. In addition to narrowing a strut according to configurations exemplified in FIGS. 7-9, a strut may also be narrowed locally by reducing its width from one side only and, for example, such narrowing may be applied to adjacent rings on an alternated side, or even within a single ring on alternating sides. All such forms of narrowing are contemplated to be within the scope of the invention.

It will be appreciated by one of ordinary skill in the art that, because the pin joints 150 in the struts 116 are configured to allow the strut to bend outwardly, away from a notional cylindrical shape occupied by the ring, that a reduction in radial thickness of the strut will have relatively more effect in permitting bending at the joint than a reduction in circumferential width of the stent and the same point. This is because the moment of inertia of the strut for bending about a circumferential axis is a function of the thickness of the strut cubed, but is a function of the width of the strut to only the first power.

Method of Manufacture

The thickness of the strut may be locally reduced by any of a variety of processes. In a first embodiment, a laser that is used to cut the pattern of the stent from a metal tube may be reduced in intensity after the desired width of the strut has been cut. The laser beam may be directed at the location of the pin joint for a short period of time which is sufficient to melt away a superficial surface of the strut at the position of the pin-joint 150, and thereby to locally reduce the thickness of the strut to the thickness, indicated in the figures, as T2, where the reduction occurs on the outside surface of the strut. Subsequent acid bathing of the entire stent may be used to smooth out any sharp discontinuities that arise from the described process of thinning. In other embodiments, the strut may be reduced in thickness by mechanically tapping the tube from which the stent will be cut, at the projected location of the pin joint. Thus, when the stent is cut, the strut will possess a reduced thickness at the desired location. Such mechanical tapping may be achieved by passing a tube, that is to be cut into a stent, into a larger diameter tube to which are attached internal flanges. An internal mandrel is passed into the bore of the stent. The mandrel is expanded to mechanically crimp a dent into the external surface of the tube. The same process may be used inside out, to place a crimped dent onto the inside diameter of the tube. This latter process has the advantage of leaving the outside surface with a smooth finish. In yet other embodiments, strut thickness may be reduced by first cutting the stent to an unmodified configuration. Then, the stent may be mounted on a mandrel to act as an anode. A wire may be wound around the stent at the center of the strut, the wire to act as a cathode. The stent thus connected to anode and cathode may then be dipped into an acid bath to undergo electrolysis. Under the application of electric potential difference in the acid bath, the wire may act to enhance cathodization of the metal at that location, and corresponding reduction in thickness to a desired extent as controlled by the period of electrolysis. In some embodiments, the reduction to the thickness of the strut may be applied to the inside surface of the strut by adjusting the laser focus depth to a point that lies beneath the outside surface of the stent, thereby ignoring material on an outside surface and removing it beneath the outside surface. This process has the advantage of leaving the outside surface with a smooth finish.

Behavior of Modified Stent

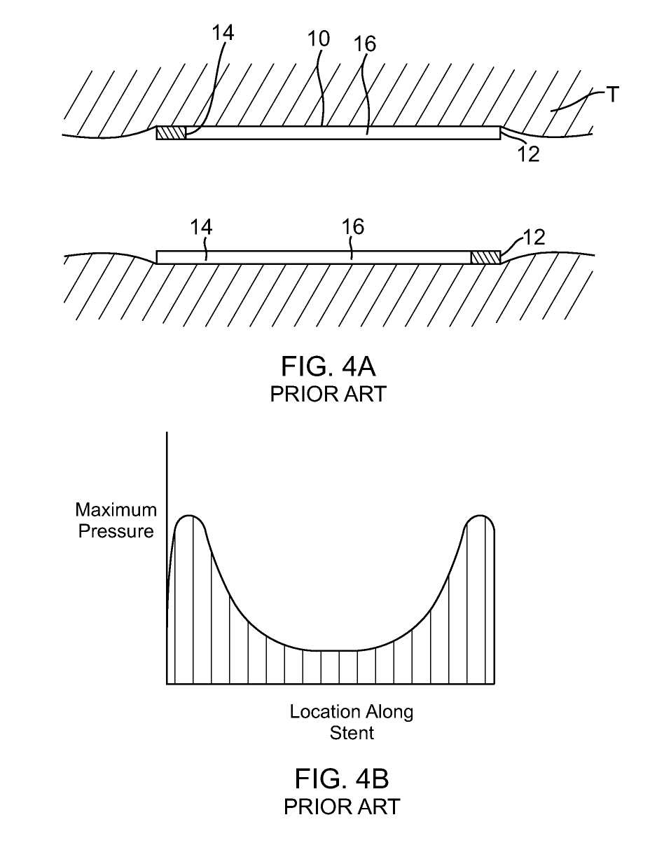

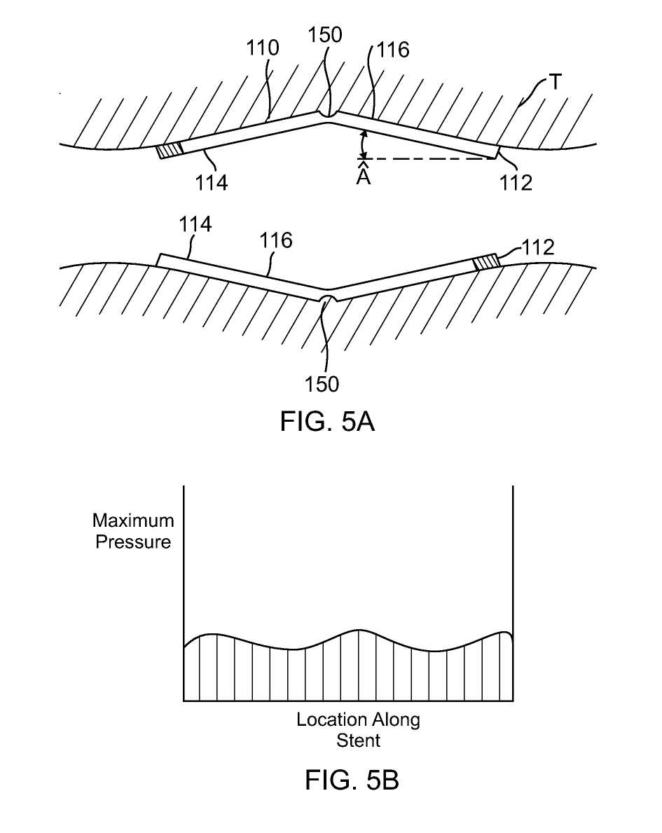

As noted above, in the prior art, with no modification of a stent to include pin joints as contemplated by the present invention, the rings 10 of the unmodified stent tend to assume a generally cylindrical shape as exemplified in FIG. 4A, and the pressure profile experienced by the vessel surrounding the stent is schematically exemplified in FIG. 4B. However, by modifying the struts 116 of a stent according to the invention to include pin joints 150 as described at the center of each strut 116 connecting a peak 112 to a valley 114 within a ring 110, each resulting ring thus modified, when expanded or permitted to self-expand, will tend to adopt a shape that departs from the generally cylindrical shape of the same stent in unmodified form. Specifically, the inclusion of pin joints 150 as described will cause each ring 110, that is so modified, to adopt an expanded shape as exemplified in FIG. 3 and FIG. 5A, where it may be seen that the pin joint 150 tends to cause the struts 116 to rotate at the location of the joint so that the strut assumes a kinked or hinged shape in which the pin joint 150 moves radially outwardly and away from a notional cylindrical form occupied by the ring 110.

The kink thus formed in the strut has the advantageous effect of more evenly distributing the pressure and forces supporting the tissue T along the length of the ring 110. For, whereas the unmodified ring 10 causes the tissue to find maximum supporting force at the extreme ends of each ring where it is supported by the peaks (at one end) and the valleys (at the other end) with little or virtually no support in the center of the strut (as schematically indicated in FIG. 4B), the kinked shape of the struts 116 of the invention provides comparatively more support in the center of each strut and hence reduces the support stress at the ends (as schematically indicated in FIG. 5B), and thus reduces the tendency of the terminal corners of the ring to dig into the tissue T. This effect may also be appreciated once it is understood that the radius of curvature of the tissue at the end corners of the ring 110 exemplified in FIG. 5A is larger than it is in relation to the embodiment of the unmodified ring 10 exemplified in FIG. 4A because the struts with pin joints tend to rotate by an angle Â at the corners, as shown in FIG. 5A, and thus support the tissue with a more gently shaped curvature. This rotation reduces the upward lift on the tissue at the corners. Following known principles of physics, the larger the radius of curvature, the smaller the stress imposed in the tissue T. Hence, introducing the pin joints 150 according to the invention gives rise to a reduction in the maximum stress experienced by the tissue of FIG. 5A, as compared with the tissue of FIG. 4A.

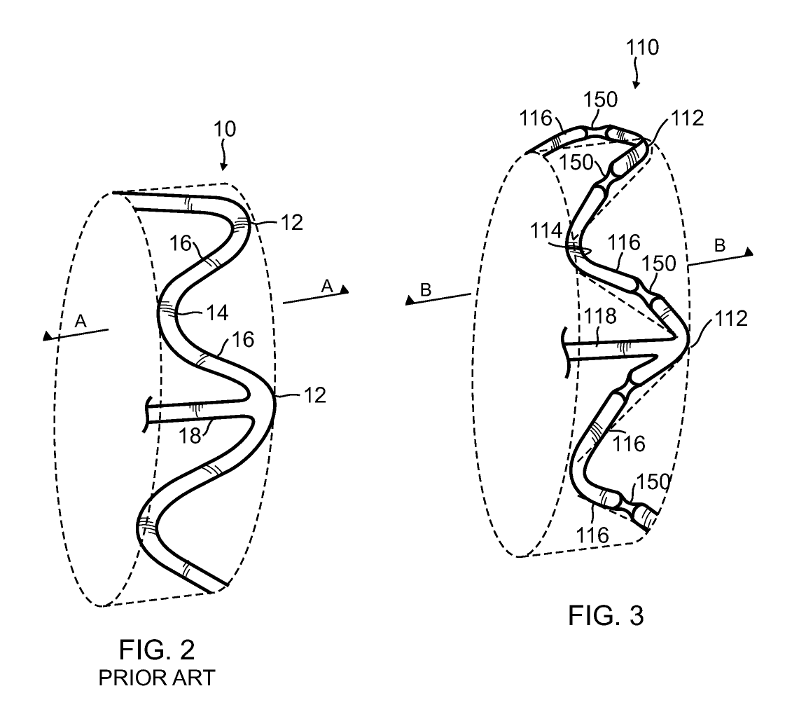

Stated differently, it may be appreciated with reference to FIG. 2 and FIG. 4A that, in unmodified form, a ring 10 (which in this example has six peaks and six valleys) will tend to provide support to vascular tissue at twelve points, namely at six valleys on the left side of the ring and at six peaks on the right side of the ring 10. However, with reference to FIG. 3 and FIG. 5A, it will be understood that outward expansion of the center of each strut 116 due to the pin joints 150 provides an additional twelve points of substantial tissue support, namely at the high point of each of the twelve pin joints 150. Thus, total scaffolding force is distributed across more points per ring, namely at twenty four points per ring 110 rather than at only twelve (in this example), and the tendency of the corners at the terminal ends to dig into the tissue and cause injury to the vessel wall is diminished.

A corollary advantage to the behavior of a ring with pin joints 150 as described, is that a balloon situated internal to the ring and configured to expand the stent, will itself be permitted to adopt a more spherical shape on the interior of the ring, while departing from a purely cylindrical shape in that region. This aspect may tend to permit the balloon also to experience a reduction in localized stresses to the extent that corners do not form a sharp edge to raise stresses in the membrane of the balloon.

In accordance with the structure of the invention set forth above, certain computer modeling, physical prototyping, and loading tests were performed to confirm the functionality of embodiments of the invention.

Confirmation of Structural Functionality by Bench Testing

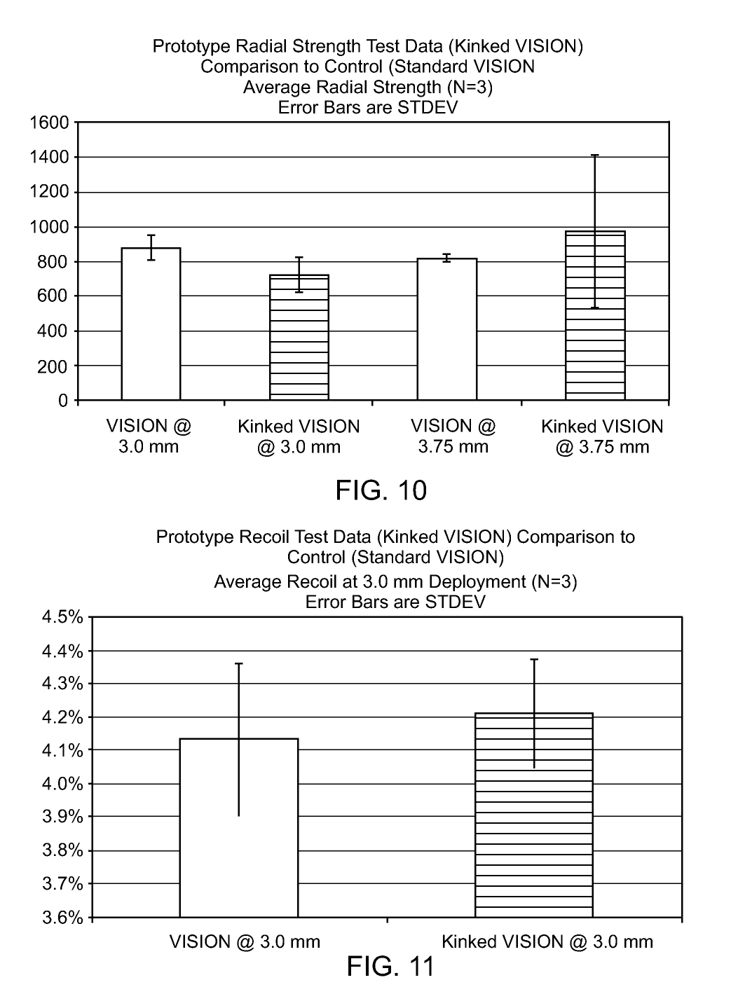

First, physical samples of actual stents were selected for bench testing. Four Vision 3.0 mm stents, were selected. During the process of manufacture, modifications were effected to two of the stents, the modifications being strut modifications to reflect pin-joints occupying about 30% of the sectional area compared with that of the unmodified struts. The length of the narrowed portion of each pin-joint was selected to be about twice the thickness of the strut. A modified and an unmodified stent were then expanded until the recommended, or “label,” diameter of 3.0 mm was reached. A second set of a modified and an unmodified stent were then expanded until the maximum post dilation diameter of 3.7 mm was reached (using a secondary inflation step with a special post-dilation balloon). Bench tests were performed on the resulting expanded stents according to known protocols, to determine the radial strength of each. The results are shown in FIG. 10, and these reflect that, for both the 3.0 mm and the 3.7 mm expanded stents, placing pin-joints as described in the center of the struts did not cause any significant loss of radial strength.

Next, the two stents (one modified, the other unmodified) that had been expanded to 3.0 mm were loaded according to known protocols to test recoil, and the results are shown in FIG. 11. Again it was found that no significant change to recoil properties was caused by introduction of the pin-joints in these stents.

Confirmation of Trauma Reduction by Computer Modeling

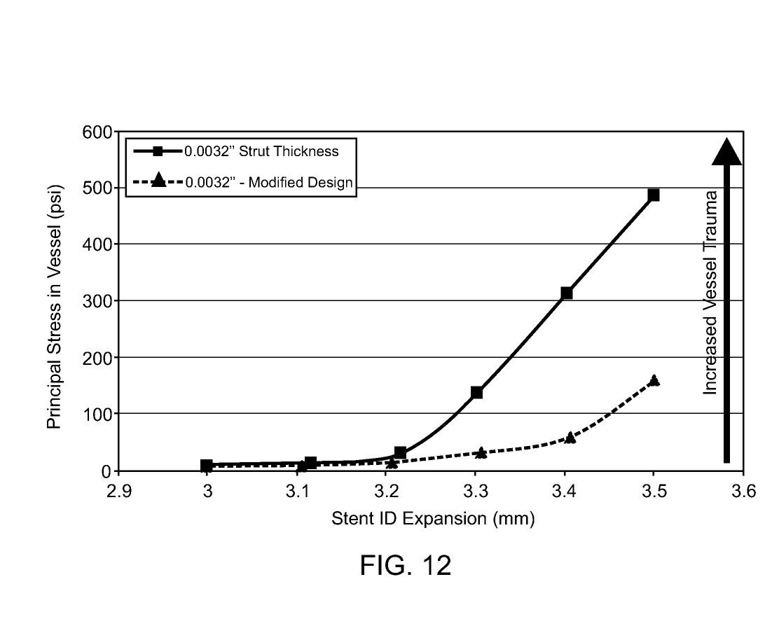

Next, the structure of two Abbott Vascular Inc. “Vision” stents each having a 3.0 mm label diameter and having a 0.0032 inch thickness were programmed into a computer for conducting known method of analysis by finite elements (also known as finite element analysis, or “FEA”). One stent model was in an unmodified form, the other was in a form modified to possess pin joints in the struts as described above. In addition to the stent in each case, a vessel was modeled to surround each stent. The vessels were modeled to possess an appropriate thickness, and relative elastic modulus of a body vessel lumen such as a blood vessel in comparison with the elastic modulus of a metal stent. Each stent model was expanded, by FEA simulation, beyond its 3.0 mm “label” diameter, and the “trauma” imparted to the vessel by each stent was recorded. The “trauma” measured in each vessel was assigned a value being the maximum principal stresses measured in the vessel. Results are shown in FIG. 12, where it is indicated that a Vision stent, modified to introduce into the struts the pin joints 150 as described above, produces a drastic reduction in maximum vessel stress when compared to the standard Vision platform. Specifically, whereas at an expanded diameter of 3.5 mm (i.e. 0.5 mm beyond label diameter) the unmodified stent produced a maximum stress of nearly 500 p.s.i, in the vessel, the modified stent at the same 3.5 mm diameter produced a maximum stress of only about 150 p.s.i. in the vessel. It will be appreciated that this is an extremely significant reduction in maximum stress, and therefore in the trauma, imparted to the vessel. Also confirmed by the FEA analysis is that the stents that were modified to possess a pin joint in the struts as described above, were found to bend radially outward at the pin joints by plastic deformation. Whereas, the balance of the strut portions, lying beyond the pin joints, were found not to exhibit outward bending by plastic deformation. Any outward deformation in the regions lying beyond the pin joints were found to bend outwardly within the elastic range. (Any plastic deformation in the peaks and troughs were found to be in the plane of the circumference of the stent, rather than outwardly and away from the plane of the circumference of the ring.)

A point may be mentioned in relation to certain known prior art. Certain stents have been described in the prior art that possess struts in which the circumferential width of the struts joining peaks and valleys are gradually narrowed, starting with no narrowing at each end of the strut where it connects to a peak or a valley respectively, and then gradually narrows toward the center point of the strut midway between peak and valley. Such prior art stents do not satisfy the limitations and do not provide the advantages of the present invention to the extent that they do not describe a reduced radial thickness of the stent, and furthermore they are not configured to compel a strut to bend only at a localized pin joint at the center of the strut. Rather, outward bending deflection in the strut may occur along its entire length. It will be understood by one of ordinary skill that, by configuring a strut having substantially uniform thickness and width to have a central pin joint with narrowing and/or thinning at only a short localization point on the strut, for flexion at that point of the strut radially away from the surface of the stent, the strut has by implication been configured to have a substantially uniform width and thickness elsewhere along the strut. This feature allows the pin jointed strut as described herein to substantially maintain its overall radial strength after being expanded, and also to retain its recoil properties as compared with its unmodified shape. By contrast, stents in the prior art as described, which have gradually tapering strut widths, are deprived of a significant quantity of the material making up the struts and, therefore, also lose significant radial strength and recoil properties. Furthermore, such prior art stents, with greater material loss, lose radiopacity, and tend to be less visible during surgical procedures.

Thus, the stent modification of the present invention provides an advantageous structure for improving the trauma characteristics of many known stent designs. The present invention may, of course, be carried out in other specific ways than those herein set forth without departing from the essential characteristics of the invention. For example, the following additional features may be applied. The stent may also be manufactured with struts and other elements that are circular, rather than rectangular. A stent may be manufactured by an additive process, or cladded, or coated in a manner that substantially increases its thickness. In such case, the pin joint may be formed by simply omitting material in that area. The present embodiments are, therefore, to be considered in all respects as illustrative and not restrictive, while the scope of the invention is set forth in the claims that follow.

I claim:

1. A system for treating a vascular condition, comprising:
a catheter configured for insertion into a vessel;
a stent mounted on the catheter and configured for delivery into the vessel, the stent comprising:
a plurality of rings, each ring being connected to an adjacent ring by at least one link, each ring including a plurality of peaks and valleys, wherein each peak is connected to an adjacent valley by a strut to provide an undulating pattern within each ring; and further wherein
each of a plurality of the struts comprises, in series, a first portion which is directly connected to a second portion, the second portion being directly connected to a third portion;
each of the first portion and the third portion has a thickness that is substantially constant throughout both the first portion and the third portion; and
the second portion has a minimum thickness between 30% and 80% of the thickness of the first and third portions, and further wherein the second portion is not coated with any therapeutic material.

2. The system of claim 1, wherein the stent is a balloon expandable stent.

3. The system of claim 1, wherein the stent is a self-expanding stent.

4. The system of claim 1, wherein the second portion has a minimum thickness between 45% and 65% of the thickness of the first and third portions.

5. The system of claim 1, wherein the second portion extends for a length not more than three times the thickness of the first and third portions.

6. The system of claim 1, wherein each of the first portion and the third portion has a width that is substantially constant throughout both the first portion and the third portion and the second portion has a minimum width that is between 30% and 80% of the width of the first and third portions.

7. The system of claim 6, wherein the second portion has a minimum width that is between 45% and 65% of the width of the first and third portions.

8. The system of claim 1, wherein the second portion is positioned on a strut, midway between a valley and a peak.

9. A system for treating a vascular condition, comprising:
a catheter configured for insertion into a vessel;
a stent mounted on the catheter and configured for delivery into the vessel, the stent comprising:
a plurality of rings, each ring being connected to an adjacent ring by at least one link, each ring including a plurality of peaks and valleys, wherein each peak is connected to an adjacent valley by a strut to provide an undulating pattern within each ring; and further wherein
each of a plurality of the struts comprises, in series, a first portion which is directly connected to a second portion, the second portion being directly connected to a third portion;
each of the first portion and the third portion has a thickness that is substantially constant throughout both the first portion and the third portion; and
each of the first portion and the third portion has a width that is substantially constant throughout both the first portion and the third portion; and
the second portion extends for a length not more than three times the thickness of the first and third portions, and wherein the second portion has a minimum width that is between 30% and 80% of the width of the first and third portions, and further wherein the second portion is not coated with any therapeutic material.

10. The system of claim 9, wherein the stent is a balloon expandable stent.

11. The system of claim 9, wherein the stent is a self-expanding stent.

12. The system of claim 9, wherein the second portion has a minimum width that is between 45% and 65% of the width of the first and third portions.

13. The system of claim 9, wherein the second portion has a minimum thickness that is between 30% and 80% of the thickness of the first and third portions.

14. The system of claim 13, wherein the second portion has a minimum thickness that is between 45% and 65% of the thickness of the first and third portions.

15. The system of claim 9, wherein the second portion is positioned on a strut midway between a valley and a peak.

16. A method of treating a condition in a vessel comprising:
mounting a stent on a catheter, wherein the stent includes a plurality of rings, each ring being connected to an adjacent ring by at least one link, each ring including a plurality of peaks and valleys, wherein each peak is connected to an adjacent valley by a strut to provide an undulating pattern within each ring, and further wherein each of a plurality of the struts comprises, in series, a first portion which is directly connected to a second portion, the second portion being directly connected to a third portion; and further wherein each of the first portion and the third portion has a thickness that is substantially constant throughout both the first portion and the third portion; wherein the second portion has a minimum thickness between 30% and 80% of the thickness of the first and third portions, and further wherein the second portion is not coated with any therapeutic material;
delivering the stent to a desired location in the vessel;
deploying the stent to an expanded condition inside the vessel; and
causing each of the plurality of struts to bend radially outwardly at the location of the second portion by causing plastic deformation outwards in the second portion while causing no plastic deformation outwards in the first and third portions.

17. The method of claim 16, wherein causing plastic deformation outwards in the second portion includes causing plastic deformation outwards over a strut length that does not exceed three times the thickness of the first and third portions.

18. The method of claim 16, wherein deploying the stent to an expanded condition includes deploying the stent by balloon.

19. The method of claim 16, wherein deploying the stent to an expanded condition includes allowing the stent to self expand.

20. The method of claim 16, wherein causing each of the plurality of struts to bend radially outwardly includes causing each of the plurality of struts to bend radially outwardly at a location midway between a valley and a peak.

21. A method of treating a condition in a vessel comprising:
mounting a stent on a catheter, wherein the stent includes a plurality of rings, each ring being connected to an adjacent ring by at least one link, each ring including a plurality of peaks and valleys, wherein each peak is connected to an adjacent valley by a strut to provide an undulating pattern within each ring; each of a plurality of the struts comprises, in series, a first portion which is directly connected to a second portion, the second portion being directly connected to a third portion; each of the first portion and the third portion has a thickness that is substantially constant throughout both the first portion and the third portion; each of the first portion and the third portion has a width that is substantially constant throughout both the first portion and the third portion; the second portion extends for a length not more than three times the thickness of the first and third portions, and wherein the second portion has a minimum width that is between 30% and 80% of the width of the first and third portions, and further wherein the second portion is not coated with any therapeutic material;
delivering the stent to a desired location in the vessel;
deploying the stent to an expanded condition inside the vessel; and
causing each of the plurality of struts to bend radially outwardly at the location of the second portion by causing plastic deformation outwards in the second portion while causing no plastic deformation outwards in the first and third portions.

22. The method of claim 21, wherein deploying the stent to an expanded condition includes deploying the stent by balloon.

23. The method of claim 21, wherein deploying the stent to an expanded condition includes allowing the stent to self expand.

24. The method of claim 21, wherein causing each of the plurality of struts to bend radially outwardly includes causing each of the plurality of struts to bend radially outwardly at a location substantially midway between a valley and a peak.

25. The method of claim 21, wherein causing each of the plurality of struts to bend radially outwardly includes causing each of the plurality of struts to bend radially outwardly at a location substantially midway between a valley and a peak.
